(19) United States
(12) Patent Application Publication
(10) Pub. No.: US 2023/0279009 A1
(43) Pub. Date: Sep. 7, 2023

(54) SOLID STATE FORMS OF AVAPRITINIB SALTS

(71) Applicant: TEVA CZECH INDUSTRIES S.R.O, Opava-Komarov (CZ)

(72) Inventors: Anantha Rajmohan Muthusamy, Sivakasi (IN); Yogesh Dhananjay Wagh, Badlapur (E) Thane (IN); Siva Rama Krishna Muppalla, Guntur (IN); Parven Kumar Luthra, Noida (IN); Meenakshi Sundaram Somasundaram, Virudu Nagar DT (IN)

(21) Appl. No.: 18/009,771

(22) PCT Filed: Jun. 15, 2021
(86) PCT No.: PCT/US2021/037359

§ 371 Date: Dec. 12, 2022

(30) Foreign Application Priority Data
Jun. 17, 2020 (IN) 202011025576
Mar. 10, 2021 (IN) 202111010120
May 26, 2021 (IN) 202111023415

(51) Int. Cl. C07D 487/04; A61P 35/00

(52) U.S. Cl. CPC C07D 487/04; A61P 35/00; C07B 2200/13

(57) ABSTRACT

The present disclosure encompasses solid state forms of Avapritinib salts and co-crystals of the hydrochloride salt of Avapritinib, processes for preparation thereof, and pharmaceutical compositions thereof as well as their pharmaceutical use, in particular in the treatment of gastrointestinal stromal tumors (GIST), solid tumors and Advanced Systemic Mastocytosis.

### FIELD OF THE DISCLOSURE

The present disclosure encompasses solid state forms of Avapritinib, in embodiments, crystalline polymorphs of Avapritinib, processes for preparation thereof, and pharmaceutical compositions thereof. Particularly, the present disclosure encompasses solid state forms of Avapritinib salts, especially crystalline forms of Avapritinib salts, processes for their preparation and pharmaceutical compositions thereof.

### BACKGROUND OF THE DISCLOSURE

Avapritinib, (1S)-1-(4-fluorophenyl)-1-[2-[4 [6-(1-methylpyrazol-4-yl)pyrrolo[2,1-f][1,2,4]triazin-4-yl]piperazin-1-yl]pyrimidin-5-yl]ethanamine, has the following chemical structure:

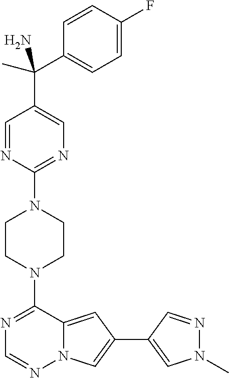 [SMILES: Cn1cc(-c2cc3c(N4CCN(c5ncc([C@@](C)(N)c6ccc(F)cc6)cn5)CC4)ncnn3c2)cn1]

Avapritinib is being developed for the treatment of gastrointestinal stromal tumors (GIST), solid tumors. Avapritinib is also under evaluation for the treatment of Advanced Systemic Mastocytosis.

The compound is described in International Publication No. WO2015/057873. International Publication Nos. WO2021/004895, WO2021/079134 and CN1121259A disclose crystalline forms of Avapritinib. International Publication No. WO2020/210669 discloses crystalline forms and salts of Avapritinib.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms and co-crystals) of Avapritinib, including Avapritinib salts.

### SUMMARY OF THE DISCLOSURE

The present disclosure provides crystalline polymorphs of Avapritinib, processes for preparation thereof, and pharmaceutical compositions thereof. Particularly, the crystalline polymorphs of Avapritinib, are salts of Avapritinib. These crystalline polymorphs can be used to prepare other solid state forms of Avapritinib, Avapritinib salts and their solid state forms.

The present disclosure also provides uses of the said solid state forms of Avapritinib, including solid state forms of Avapritinib salts, in the preparation of other solid state forms of Avapritinib or salts thereof.

The present disclosure provides crystalline polymorphs of Avapritinib including Avapritinib salts, for use in medicine, including for the treatment of gastrointestinal stromal tumors (GIST), solid tumors, and Advanced Systemic Mastocytosis, preferably gastrointestinal stromal tumors (GIST) and solid tumors, and more preferably gastrointestinal stromal tumors (GIST).

The present disclosure also encompasses the use of crystalline polymorphs of Avapritinib, including Avapritinib salts, of the present disclosure for the preparation of pharmaceutical compositions and/or formulations.

In another aspect, the present disclosure provides pharmaceutical compositions comprising crystalline polymorphs of Avapritinib according to the present disclosure.

The present disclosure includes processes for preparing the above mentioned pharmaceutical compositions. The processes include combining any one or a combination of the crystalline polymorphs of Avapritinib with at least one pharmaceutically acceptable excipient.

The crystalline polymorphs of Avapritinib, including Avapritinib salts, as defined herein and the pharmaceutical compositions or formulations of the crystalline polymorphs of Avapritinib, including Avapritinib salts, may be used as medicaments, such as for the treatment of gastrointestinal stromal tumors (GIST), solid tumors, and Advanced Systemic Mastocytosis, preferably gastrointestinal stromal tumors (GIST) and solid tumors, and more preferably gastrointestinal stromal tumors (GIST).

The present disclosure also provides methods of treating gastrointestinal stromal tumors (GIST), by administering a therapeutically effective amount of any one or a combination of the crystalline polymorphs of Avapritinib, including Avapritinib salts, of the present disclosure, or at least one of the above pharmaceutical compositions, to a subject suffering from gastrointestinal stromal tumours (GIST), solid tumors, and Advanced Systemic Mastocytosis, preferably gastrointestinal stromal tumors (GIST) and solid tumors, and more preferably gastrointestinal stromal tumors (GIST) or otherwise in need of the treatment.

The present disclosure also provides uses of crystalline polymorphs of Avapritinib, including Avapritinib salts, of the present disclosure, or at least one of the above pharmaceutical compositions, for the manufacture of medicaments for treating e.g., gastrointestinal stromal tumours (GIST), solid tumors, and Advanced Systemic Mastocytosis, preferably gastrointestinal stromal tumors (GIST) and solid tumors, and more preferably gastrointestinal stromal tumors (GIST).

### BRIEF DESCRIPTION OF THE DRAWINGS

FIG. 1 shows a characteristic X-ray powder diffraction pattern (XRPD) of Form AC1 of Avapritinib: benzoic acid.

FIG. 2 shows a characteristic X-ray powder diffraction pattern (XRPD) of Form AC2 of Avapritinib: succinic acid.

FIG. 3 shows a characteristic X-ray powder diffraction pattern (XRPD) of Form AC3 of Avapritinib: fumaric acid.

FIG. 4 shows a characteristic X-ray powder diffraction pattern (XRPD) of Form AC4 of Avapritinib: adipic acid.

FIG. 5 shows a characteristic X-ray powder diffraction pattern (XRPD) of Form AC6 of Avapritinib: salicylic acid.

FIG. 6 shows a characteristic X-ray powder diffraction pattern (XRPD) of Form AC7 of Avapritinib: (R)-mandelic acid.

FIG. 7 shows a characteristic X-ray powder diffraction pattern (XRPD) of Form AC8 of Avapritinib: benzoic acid.

FIG. 8 shows a characteristic X-ray powder diffraction pattern (XRPD) of Form AC9 of Avapritinib: benzoic acid.

FIG. 9 shows a characteristic X-ray powder diffraction pattern (XRPD) of Form AC10 of Avapritinib: cinnamic acid.

FIG. 10 shows a characteristic X-ray powder diffraction pattern (XRPD) of Form AC11 of Avapritinib: p-hydroxybenzoic acid.

FIG. 11 shows a characteristic X-ray powder diffraction pattern (XRPD) of Form AT1 of Avapritinib.

FIG. 12 shows a characteristic X-ray powder diffraction pattern (XRPD) of Form AT5 of Avapritinib.

FIG. 13 shows a characteristic X-ray powder diffraction pattern (XRPD) of co-crystal of Avapritinib HCl salt and benzoic acid-Form AHC1.

FIG. 14 shows a characteristic X-ray powder diffraction pattern (XRPD) of co-crystal of Avapritinib HCl salt and p-hydroxybenzoic acid-Form AHC2.

FIG. 15 shows a characteristic X-ray powder diffraction pattern (XRPD) of co-crystal of Avapritinib HCl salt and cinnamic acid-Form AHC3.

### DETAILED DESCRIPTION OF THE DISCLOSURE

The present disclosure encompasses solid state forms of Avapritinib, including crystalline polymorphs of Avapritinib, processes for preparation thereof, and pharmaceutical compositions thereof.

Solid state properties of Avapritinib and crystalline polymorphs thereof can be influenced by controlling the conditions under which Avapritinib and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Avapritinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Avapritinib. In some embodiments of the disclosure, the described crystalline polymorph of Avapritinib may contain from about 1% to about 20% (w/w), from about 5% to about 20% (w/w), or from about 5% to about 10% (w/w) of one or more other crystalline polymorph of the same Avapritinib.

Depending on which other crystalline polymorphs a comparison is made, the crystalline polymorphs of Avapritinib of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density. The crystalline polymorphs of the present disclosure may, in particular, be stable to stress conditions such as: grinding, pressure, heating, and/or exposure to humidity, and may have advantageous solubility characteristics at physiologically relevant pH values. In addition, The crystalline polymorphs of the present disclosure, in particular the polymorphs of Avapritinib salts, may be used as intermediates for the purification of Avapritinib.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Avapritinib referred to herein as being characterized by graphical data “as depicted in” or “as substantially depicted in” a Figure will thus be understood to include any crystal forms of Avapritinib characterized with the graphical data having such small variations, as are well known to the skilled person, in comparison with the Figure.

As used herein, and unless stated otherwise, the term “anhydrous” in relation to crystalline forms of Avapritinib, relates to a crystalline form of Avapritinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “Co-crystal” as used herein is defined as a crystalline material including two or more molecules in the same crystalline lattice and associated by non-ionic and non-covalent bonds. In some embodiments, the co-crystal includes two or more molecules which are in natural state.

As used herein, the term “isolated” in reference to crystalline polymorph of Avapritinib of the present disclosure corresponds to a crystalline polymorph of Avapritinib that is physically separated from the reaction mixture in which it is formed.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

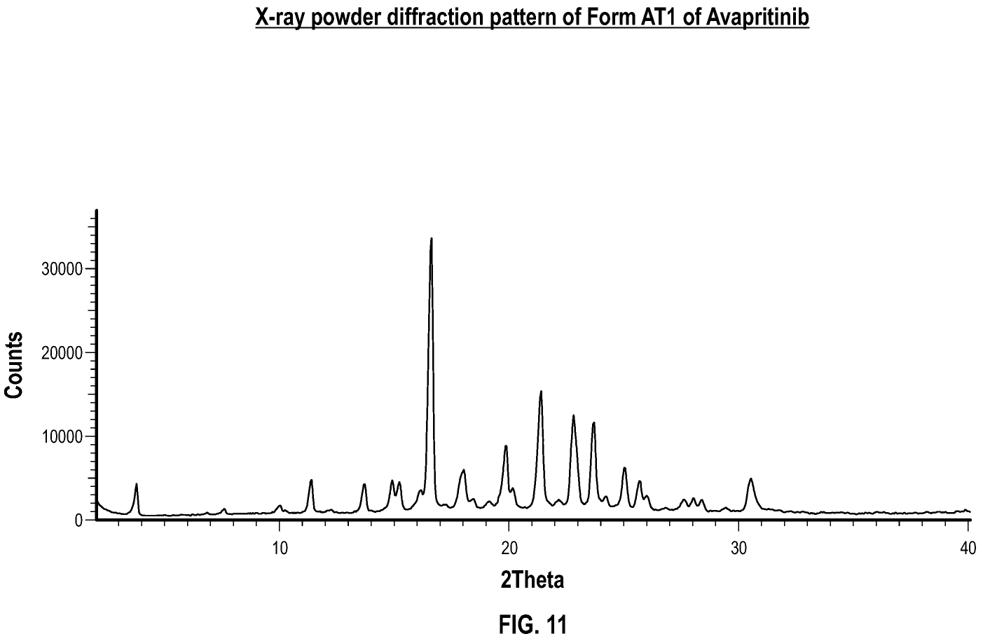

As used herein, Form AT1 of Avapritinib refers to a crystalline form of Avapritinib having an X-ray powder diffraction pattern having characteristic peaks at 3.8, 16.6, 21.4, 22.8 and 23.7 degrees 2-theta±0.2 degrees 2-theta, and optionally having any one, two, three, four or five additional peaks selected from 11.4, 13.7, 19.9, 25.1 and 30.5 degrees 2-theta±0.2 degrees 2-theta. Form AT1 of Avapritinib may particularly refer to a crystalline form having an XRPD substantially as depicted in FIG. 11. Form AT1 of Avapritinib may be prepared according to Example 1 herein.

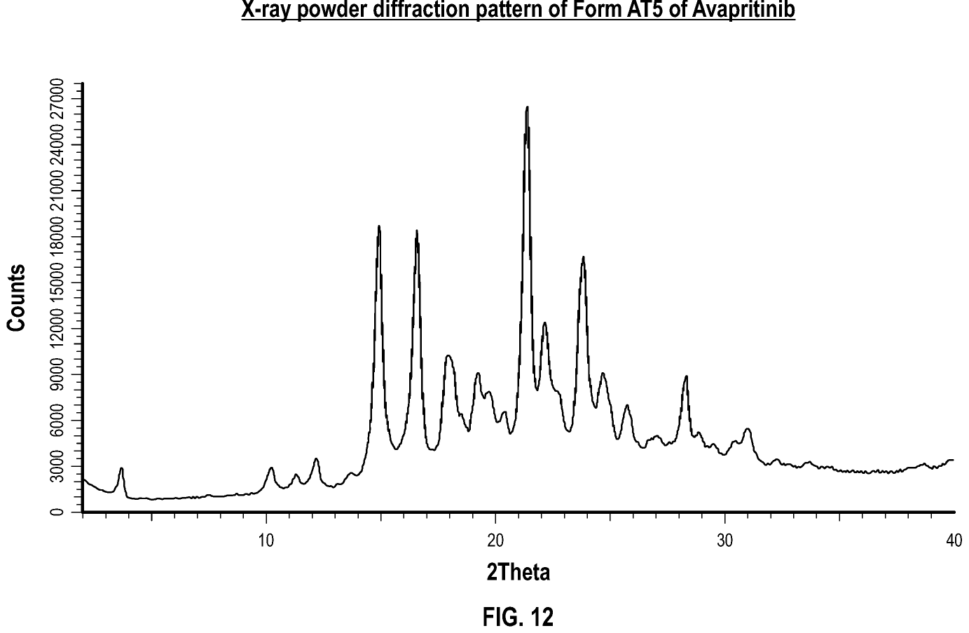

As used herein, Form AT5 of Avapritinib refers to a crystalline form of Avapritinib having an X-ray powder diffraction pattern having characteristic peaks at 10.2, 12.1, 14.8, 22.1 and 24.6 degrees 2-theta±0.2 degrees 2-theta, and optionally having any one, two or three additional peaks selected from 3.6, 19.2 and 28.3 degrees 2-theta±0.2 degrees 2-theta. Form AT5 of Avapritinib may particularly refer to a crystalline form having an XRPD substantially as depicted in FIG. 12. Form AT5 of Avapritinib may be prepared according to Example 2 herein.

The present disclosure includes a crystalline polymorph of Avapritinib: benzoic acid, designated Form AC1.

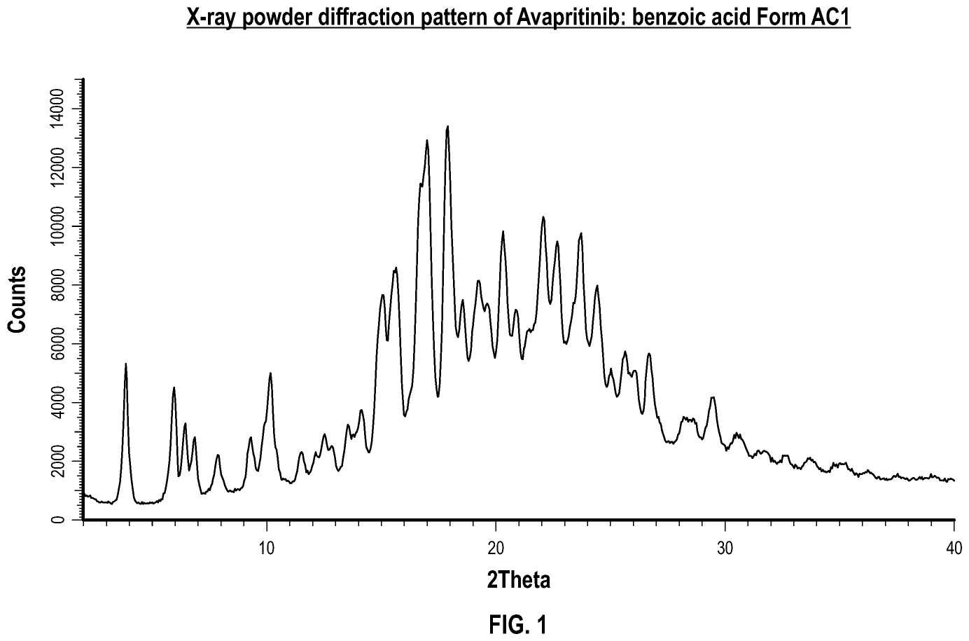

Crystalline Form AC1 of Avapritinib: benzoic acid may be characterized by data selected from one or more of the following: an X-ray powder diffraction pattern substantially as depicted in FIG. 1; an X-ray powder diffraction pattern having peaks at 5.9, 6.4, 10.1, 15.5 and 22.0 degrees 2-theta±0.2 degrees 2-theta; and combinations of these data.

Crystalline Form AC1 of Avapritinib: benzoic acid may be further characterized by an X-ray powder diffraction pattern having peaks at 5.9, 6.4, 10.1, 15.5 and 22.0 degrees 2-theta±0.2 degrees 2-theta, and also having any one, two or three additional peaks selected from 9.2, 17.8 and 26.6 degrees 2-theta±0.2 degrees 2-theta.

Crystalline Form AC1 of Avapritinib: benzoic acid may be further characterized by an X-ray powder diffraction pattern having peaks at 5.9, 6.4, 9.2, 10.1, 15.5, 17.8, 22.0, and 26.6 degrees 2-theta±0.2 degrees 2-theta.

The molar ratio between Avapritinib and benzoic acid may be 1:1.

Crystalline Avapritinib: benzoic acid may be a co-crystal of Avapritinib and benzoic acid. Alternatively, crystalline Avapritinib: benzoic acid may be a salt. Preferably, crystalline Avapritinib: benzoic acid according to the invention is a salt of Avapritinib with benzoic acid (Avapritinib benzoate).

The present disclosure comprises also a crystalline polymorph of Avapritinib: succinic acid, designated Form AC2.

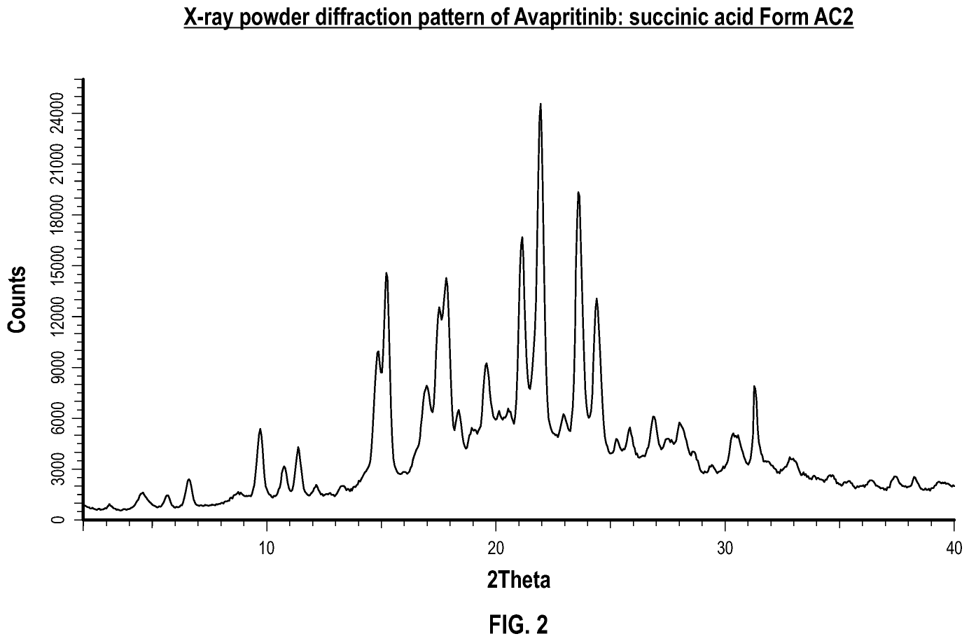

Crystalline Form AC2 of Avapritinib: succinic acid may be characterized by data selected from one or more of the following: an X-ray powder diffraction pattern substantially as depicted in FIG. 2; an X-ray powder diffraction pattern having peaks at 9.8, 10.8, 22.0, 24.5 and 27.0 degrees 2-theta±0.2 degrees 2-theta; and combinations of these data.

Crystalline Form AC2 of Avapritinib: succinic acid may be further characterized by an X-ray powder diffraction pattern having peaks at 9.8, 10.8, 22.0, 24.5 and 27.0 degrees 2-theta±0.2 degrees 2-theta, and also having any one, two, three or four additional peaks selected from 6.7, 11.4, 19.7 and 23.7 degrees 2-theta±0.2 degrees 2-theta.

Crystalline Form AC2 of Avapritinib: succinic acid may be further characterized by an X-ray powder diffraction pattern having peaks at 6.7, 9.8, 10.8, 11.4, 19.7, 22.0, 23.7, 24.5 and 27.0 degrees 2-theta±0.2 degrees 2-theta.

Crystalline Avapritinib: succinic acid may be a co-crystal of Avapritinib and succinic acid. Alternatively, crystalline Avapritinib: succinic acid may be a salt. Preferably, crystalline Avapritinib: succinic acid according to the invention is a salt of Avapritinib with succinic acid (Avapritinib succinate).

The ratio between Avapritinib and succinic acid may be 1:1.

The present disclosure includes a crystalline polymorph of Avapritinib: fumaric acid designated Form AC3.

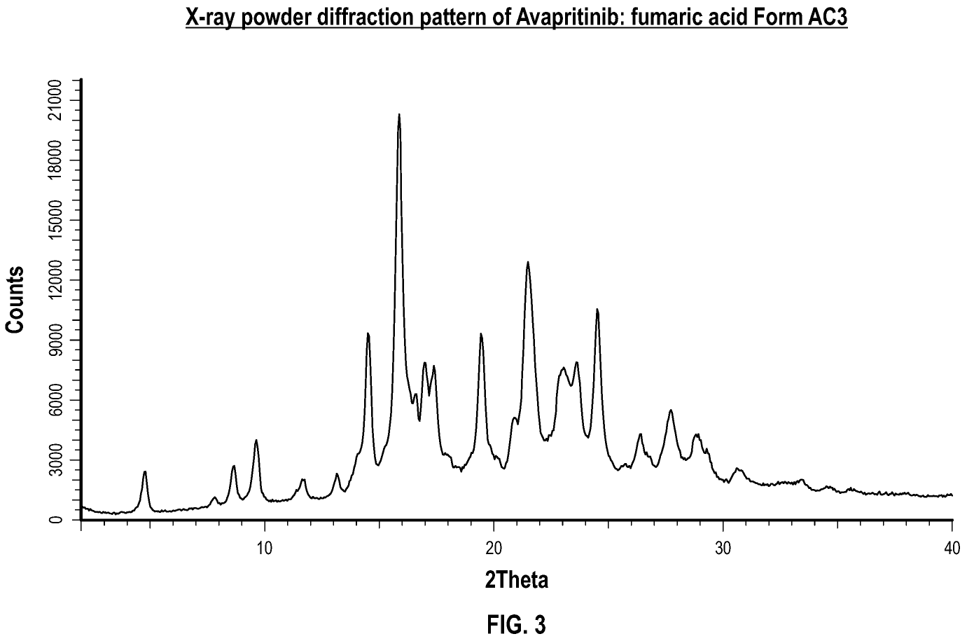

Crystalline Form AC3 of Avapritinib: fumaric acid may be characterized by data selected from one or more of the following: an X-ray powder diffraction pattern substantially as depicted in FIG. 3; an X-ray powder diffraction pattern having peaks at 4.8, 9.6, 14.5, 15.9 and 19.4 degrees 2-theta±0.2 degrees 2-theta; and combinations of these data.

Crystalline Form AC3 of Avapritinib: fumaric acid may be further characterized by an X-ray powder diffraction pattern having peaks at 4.8, 9.6, 14.5, 15.9 and 19.4 degrees 2-theta±0.2 degrees 2-theta, and also having any one, two or three additional peaks selected from 8.7, 21.4 and 24.5 degrees 2-theta±0.2 degrees 2-theta.

Crystalline Form AC3 of Avapritinib: fumaric acid may be further characterized by an X-ray powder diffraction pattern having peaks at 4.8, 8.7, 9.6, 14.5, 15.9, 19.4, 21.4 and 24.5 degrees 2-theta±0.2 degrees 2-theta.

Crystalline Avapritinib: fumaric acid may be a co-crystal of Avapritinib and fumaric acid. Alternatively, crystalline Avapritinib: fumaric acid may be a salt. Preferably, crystalline Avapritinib: fumaric acid according to the invention is a salt of Avapritinib with fumaric acid (Avapritinib fumarate, preferably Avapritinib hemifumarate).

The ratio between Avapritinib and fumaric acid ratio may be 1:0.5.

The present disclosure comprises also a crystalline polymorph of Avapritinib: adipic acid, designated Form AC4.

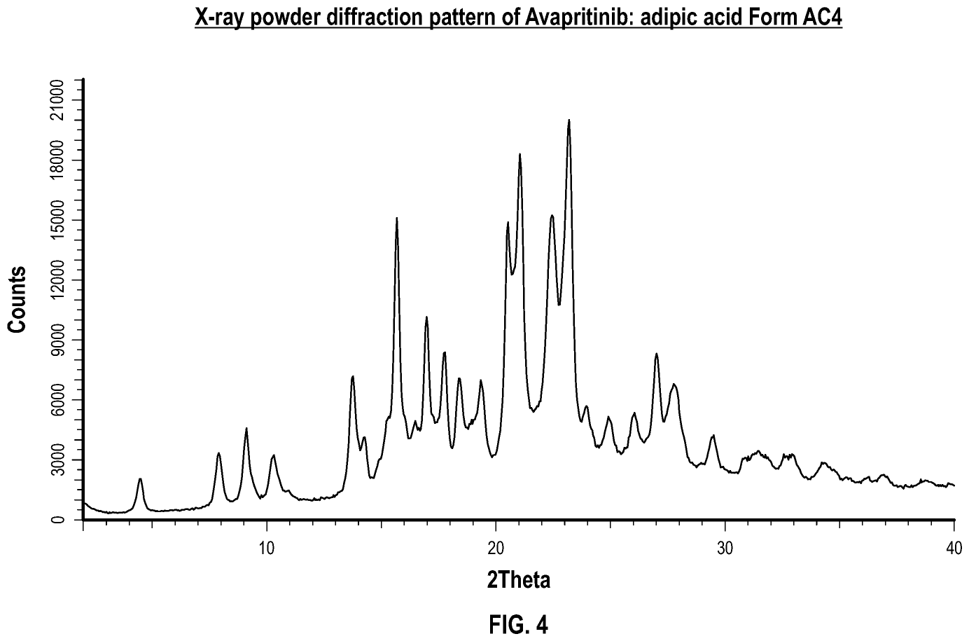

Crystalline Form AC4 of Avapritinib: adipic acid may be characterized by data selected from one or more of the following: an X-ray powder diffraction pattern substantially as depicted in FIG. 4; an X-ray powder diffraction pattern having peaks at 8.1, 9.3, 10.4, 15.9 and 23.4 degrees 2-theta±0.2 degrees 2-theta; and combinations of these data.

Crystalline Form AC4 of Avapritinib: adipic acid may be further characterized by an X-ray powder diffraction pattern having peaks at 8.1, 9.3, 10.4, 15.9 and 23.4 degrees 2-theta±0.2 degrees 2-theta, and also having any one, two, three or four additional peaks selected from 13.9, 17.2, 21.2 and 27.2 degrees 2-theta±0.2 degrees 2-theta.

Crystalline Form AC4 of Avapritinib: adipic acid may be further characterized by an X-ray powder diffraction pattern having peaks at 8.1, 9.3, 10.4, 13.9, 15.9, 17.2, 21.2, 23.4, and 27.2 degrees 2-theta±0.2 degrees 2-theta.

Crystalline Avapritinib: adipic acid may be a co-crystal of Avapritinib and adipic acid. Alternatively, crystalline Avapritinib: adipic acid may be a salt. Preferably, crystalline Avapritinib: adipic acid according to the invention is a salt of Avapritinib with adipic acid (Avapritinib adipate, preferably Avapritinib hemiadipate).

The ratio between Avapritinib and adipic acid may be 1:0.5.

The present disclosure comprises a crystalline polymorph of Avapritinib: salicylic acid, designated Form AC6.

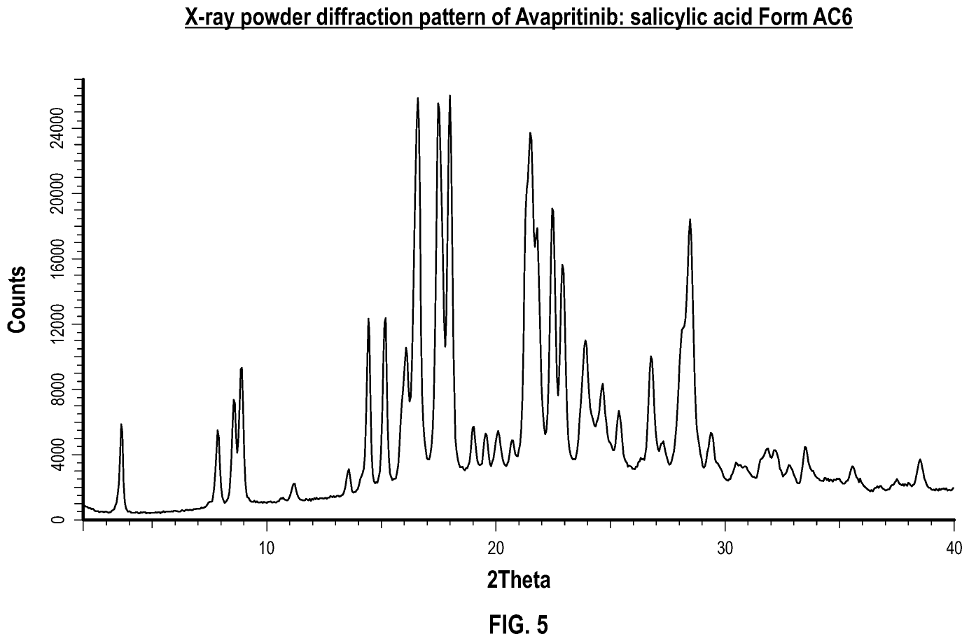

Crystalline Form AC6 of Avapritinib: salicylic acid may be characterized by data selected from one or more of the following: an X-ray powder diffraction pattern substantially as depicted in FIG. 5; an X-ray powder diffraction pattern having peaks at 8.0, 9.0, 14.6, 17.6 and 26.9 degrees 2-theta±0.2 degrees 2-theta; and combinations of these data.

Crystalline Form AC6 of Avapritinib: salicylic acid may be further characterized by an X-ray powder diffraction pattern having peaks at 8.0, 9.0, 14.6, 17.6 and 26.9 degrees 2-theta±0.2 degrees 2-theta, and also having any one, two, three or four additional peaks selected from 15.3, 18.1, 22.6 and 23.0 degrees 2-theta±0.2 degrees 2-theta.

Crystalline Form AC6 of Avapritinib: salicylic acid may be further characterized by an X-ray powder diffraction pattern having peaks at 8.0, 9.0, 14.6, 15.3, 17.6, 18.1, 22.6, 23.0, and 26.9 degrees 2-theta±0.2 degrees 2-theta.

Crystalline Avapritinib: salicylic acid may be a co-crystal of Avapritinib and salicylic acid. Alternatively, crystalline Avapritinib: salicylic acid may be a salt. Preferably, crystalline Avapritinib: salicylic acid according to the invention is a salt of Avapritinib with salicylic acid (Avapritinib salicylate).

The present disclosure further comprises a crystalline polymorph of Avapritinib: (R)-mandelic acid, designated Form AC7.

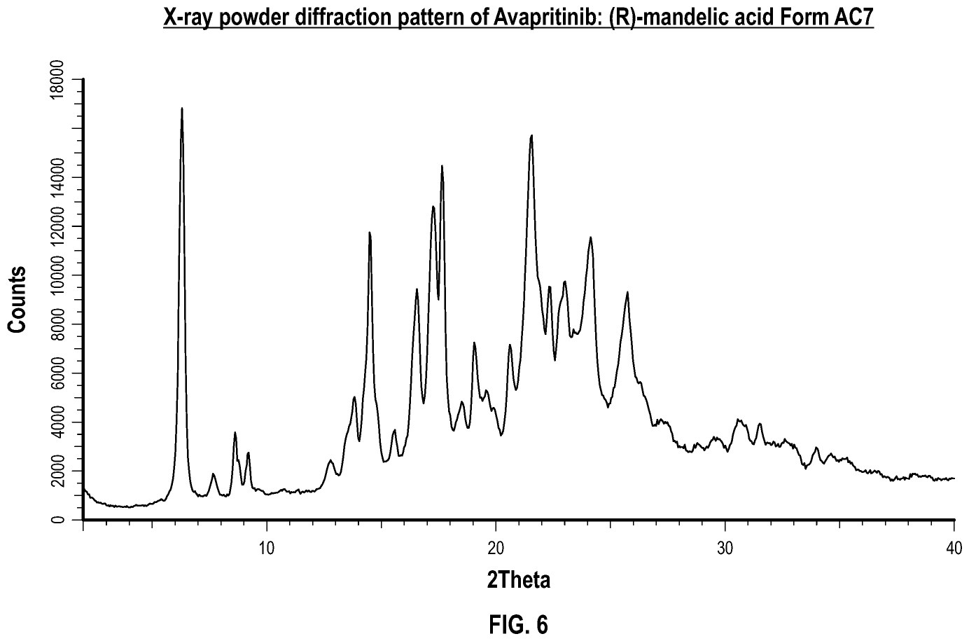

Crystalline Form AC7 of Avapritinib: (R)-mandelic acid may be characterized by data selected from one or more of the following: an X-ray powder diffraction pattern substantially as depicted in FIG. 6; an X-ray powder diffraction pattern having peaks at 6.4, 8.7, 9.2, 14.5 and 17.7 degrees 2-theta±0.2 degrees 2-theta; and combinations of these data.

Crystalline Form AC7 of Avapritinib: (R)-mandelic acid may be further characterized by an X-ray powder diffraction pattern having peaks at 6.4, 8.7, 9.2, 14.5 and 17.7 degrees 2-theta±0.2 degrees 2-theta, and also having any one, two or three additional peaks selected from 19.1, 24.1 and 25.8 degrees 2-theta±0.2 degrees 2-theta.

Crystalline Form AC7 of Avapritinib: (R)-mandelic acid may be further characterized by an X-ray powder diffraction pattern having peaks at 6.4, 8.7, 9.2, 14.5, 17.7, 19.1, 24.1 and 25.8 degrees 2-theta±0.2 degrees 2-theta.

Crystalline Avapritinib: (R)-mandelic acid may be a co-crystal of Avapritinib and R-mandelic acid. Alternatively, crystalline Avapritinib: (R)-mandelic acid may be a salt. Preferably, crystalline Avapritinib: (R)-mandelic acid according to the invention is a salt of Avapritinib with (R)-mandelic acid (Avapritinib (R)-mandelate).

In a further embodiment, the present disclosure further comprises a crystalline polymorph of Avapritinib: benzoic acid designated Form AC8.

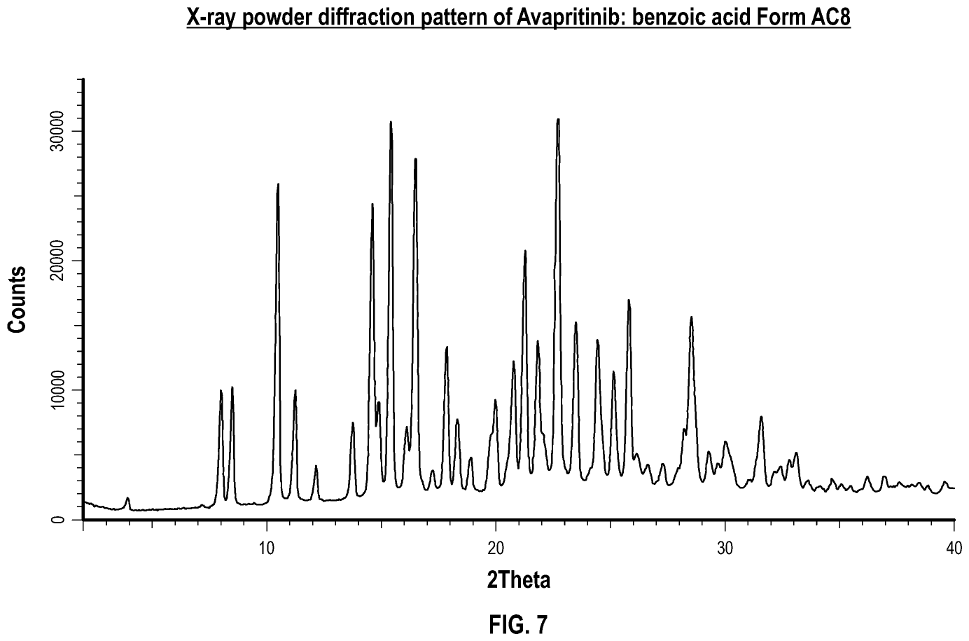

Crystalline Form AC8 of Avapritinib: benzoic acid may be characterized by data selected from one or more of the following: an X-ray powder diffraction pattern substantially as depicted in FIG. 7; an X-ray powder diffraction pattern having peaks at 8.0, 8.5, 11.2, 14.6 and 31.6 degrees 2-theta±0.2 degrees 2-theta; and combinations of these data.

Crystalline Form AC8 of Avapritinib: benzoic acid may be further characterized by an X-ray powder diffraction pattern having peaks at 8.0, 8.5, 11.2, 14.6 and 31.6 degrees 2-theta±0.2 degrees 2-theta, and also having any one, two or three additional peaks selected from 10.4, 16.5 and 23.5 degrees 2-theta±0.2 degrees 2-theta.

Crystalline Form AC8 of Avapritinib: benzoic acid may be further characterized by an X-ray powder diffraction pattern having peaks at 8.0, 8.5, 10.4, 11.2, 14.6, 16.5 and 23.5 and 31.6 degrees 2-theta±0.2 degrees 2-theta.

The molar ratio between Avapritinib and benzoic acid may be 1:1.

Crystalline Form AC8 of Avapritinib: benzoic acid may be a co-crystal of Avapritinib and benzoic acid. Alternatively, crystalline Avapritinib: benzoic acid may be a salt. Preferably, crystalline Avapritinib: benzoic acid according to the invention is a salt of Avapritinib with benzoic acid (Avapritinib benzoate).

The present disclosure also provides a crystalline polymorph of Avapritinib: benzoic acid designated Form AC9.

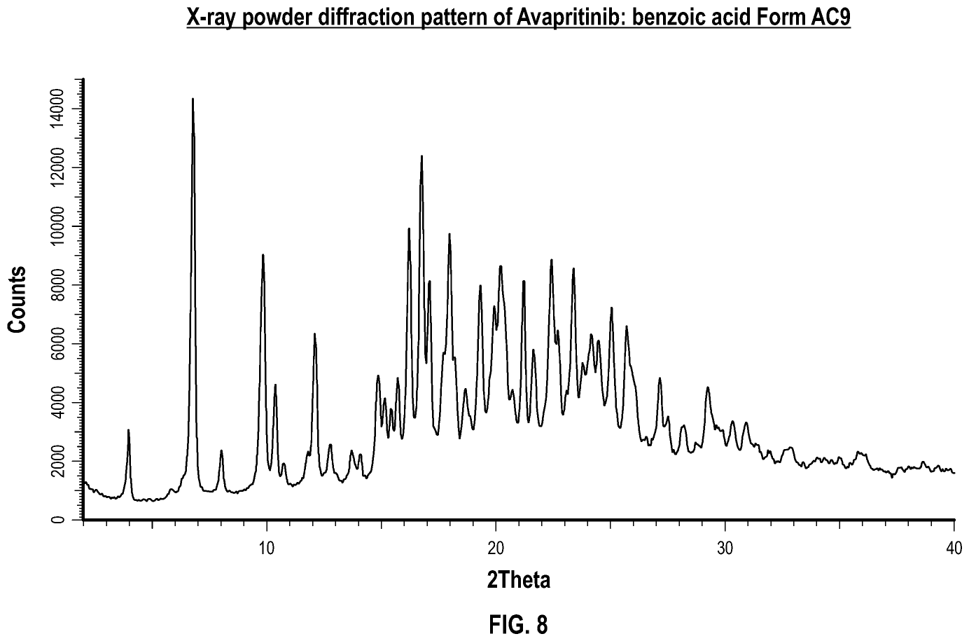

Crystalline Form AC9 of Avapritinib: benzoic acid may be characterized by data selected from one or more of the following: an X-ray powder diffraction pattern substantially as depicted in FIG. 8; an X-ray powder diffraction pattern having peaks at 4.0, 9.8, 16.2, 16.8 and 23.4 degrees 2-theta±0.2 degrees 2-theta; and combinations of these data.

Crystalline Form AC9 of Avapritinib: benzoic acid may be further characterized by an X-ray powder diffraction pattern having peaks at 4.0, 9.8, 16.2, 16.8 and 23.4 degrees 2-theta±0.2 degrees 2-theta, and also having any one, two, three or four additional peaks selected from 6.8, 18.0, 19.3 and 22.4 degrees 2-theta±0.2 degrees 2-theta.

Crystalline Form AC9 of Avapritinib: benzoic acid may be further characterized by an X-ray powder diffraction pattern having peaks at 4.0, 6.8, 9.8, 16.2, 16.8, 18.0, 19.3, 22.4, and 23.4 degrees 2-theta±0.2 degrees 2-theta.

The molar ratio between Avapritinib and benzoic acid may be 1:1.

Crystalline Form AC9 of Avapritinib: benzoic acid may be a co-crystal of Avapritinib and benzoic acid. Alternatively, crystalline Avapritinib: benzoic acid may be a salt. Preferably, crystalline Avapritinib: benzoic acid according to the invention is a salt of Avapritinib with benzoic acid (Avapritinib benzoate).

The present disclosure comprises a crystalline polymorph of Avapritinib: cinnamic acid, designated Form AC10.

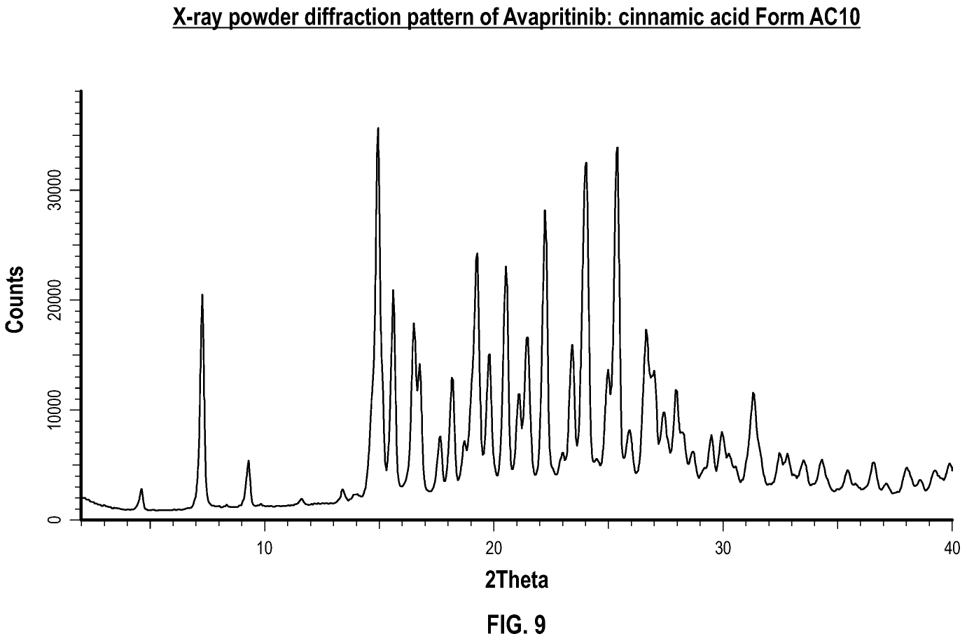

Crystalline Form AC10 of Avapritinib: cinnamic acid may be characterized by data selected from one or more of the following: an X-ray powder diffraction pattern substantially as depicted in FIG. 9; an X-ray powder diffraction pattern having peaks at 9.3, 15.6, 22.2, 24.0 and 25.4 degrees 2-theta±0.2 degrees 2-theta; and combinations of these data.

Crystalline Form AC10 of Avapritinib: cinnamic acid may be further characterized by an X-ray powder diffraction pattern having peaks at 9.3, 15.6, 22.2, 24.0 and 25.4 degrees 2-theta±0.2 degrees 2-theta, and also having any one, two, three, four or five additional peaks selected from 4.6, 7.3, 14.9, 17.6 and 19.8 degrees 2-theta±0.2 degrees 2-theta.

Crystalline Form AC10 of Avapritinib: cinnamic acid may be further characterized by an X-ray powder diffraction pattern having peaks at 4.6, 7.3, 9.3, 14.9, 15.6, 17.6, 19.8, 22.2, 24.0, and 25.4 degrees 2-theta±0.2 degrees 2-theta.

The molar ratio between Avapritinib and cinnamic acid may be 1:1.

Crystalline Form AC10 of Avapritinib: cinnamic acid may be a co-crystal of Avapritinib and cinnamic acid. Alternatively, crystalline Avapritinib: cinnamic acid may be a salt. Preferably, crystalline Avapritinib: cinnamic acid according to the invention is a salt of Avapritinib with cinnamic acid (Avapritinib cinnamate).

The present disclosure also provides a crystalline polymorph of Avapritinib: p-hydroxybenzoic acid designated Form AC11.

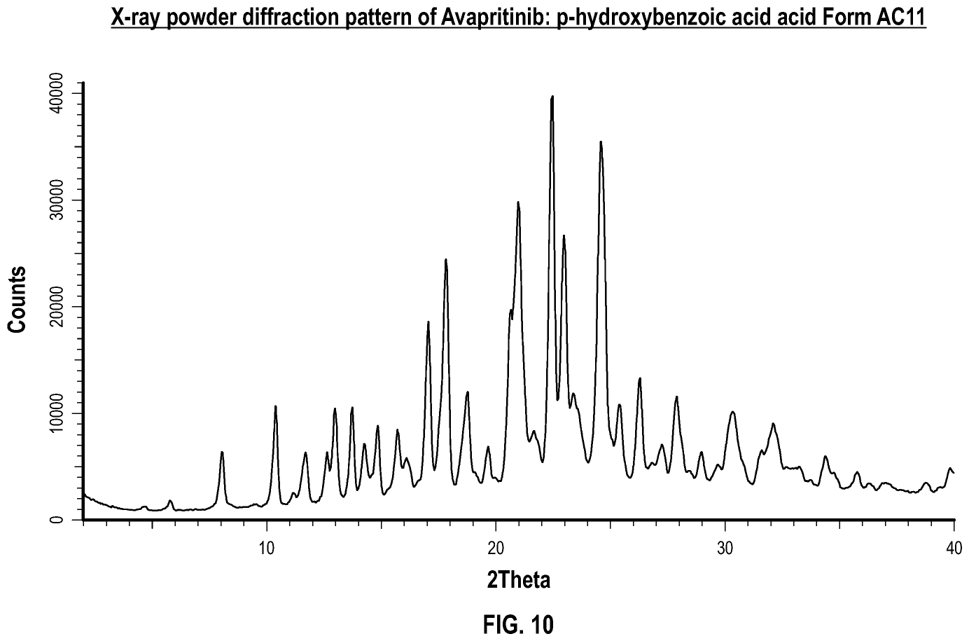

Crystalline Form AC11 of Avapritinib: p-hydroxybenzoic acid may be characterized by data selected from one or more of the following: an X-ray powder diffraction pattern substantially as depicted in FIG. 10; an X-ray powder diffraction pattern having peaks at 13.0, 13.7, 17.0, 24.6 and 26.3 degrees 2-theta±0.2 degrees 2-theta; and combinations of these data.

Crystalline Form AC11 of Avapritinib: p-hydroxybenzoic acid may be further characterized by an X-ray powder diffraction pattern having peaks at 13.0, 13.7, 17.0, 24.6 and 26.3 degrees 2-theta±0.2 degrees 2-theta, and also having any one, two, three or four additional peaks selected from 8.0, 10.4, 11.7 and 18.7 4 degrees 2-theta±0.2 degrees 2-theta.

Alternatively, crystalline Form AC11 of Avapritinib: p-hydroxybenzoic acid may be further characterized by an X-ray powder diffraction pattern having peaks at 13.0, 13.7, 17.0, 24.6 and 26.3 degrees 2-theta±0.2 degrees 2-theta, and also having any one, two, three or four additional peaks selected from 8.0, 10.4, 11.7 and 18.7 degrees 2-theta±0.2 degrees 2-theta.

Crystalline Form AC11 of Avapritinib: p-hydroxybenzoic acid may be further characterized by an X-ray powder diffraction pattern having peaks at 8.0, 10.4, 11.7, 13.0, 13.7, 17.0, 18.7, 24.6, and 26.3 degrees 2-theta±0.2 degrees 2-theta.

The molar ratio between Avapritinib and p-hydroxybenzoic acid may be 1:1.

Crystalline Form AC11 of Avapritinib: p-hydroxybenzoic acid may be a co-crystal of Avapritinib and p-hydroxybenzoic acid. Alternatively, crystalline Avapritinib: p-hydroxybenzoic acid may be a salt. Preferably, crystalline Avapritinib: p-hydroxybenzoic acid according to the invention is a salt of Avapritinib with p-hydroxybenzoic acid (Avapritinib p-hydroxybenzoate).

Solid state forms AC1 to AC11 may be prepared by crystallization from a solvent comprising Avapritinib and the appropriate carboxylic acid. The process may comprise preparing a mixture of Avapritinib and a carboxylic acid in a solvent, and crystallising the Avapritinib salt.

Particularly, the solid state forms AC1 to AC11 may be prepared by a process comprising:
(a) combining Avapritinib with a carboxylic acid;
(b) adding a solvent; and
(c) crystallization of the Avapritinib salt.

In step (a) the acid is selected from: benzoic acid, succinic acid, fumaric acid, adipic acid, salicylic acid, (R)-mandelic acid, cinnamic acid and p-hydroxybenzoic acid. In a preferred embodiment, the acid is selected from: benzoic acid, cinnamic acid and p-hydroxybenzoic acid.

In any embodiment of the process, the carboxylic acid (particularly benzoic acid, succinic acid, salicylic acid, (R)-mandelic acid, cinnamic acid, and hydroxybenzoic acid) may be used in an amount of 0.99 to about 1.1 equivalents relative to Avapritinib, and preferably about 1 equivalent relative to Avapritinib. Preferably, fumaric acid and adipic acid may be used in an amount of about 0.49 to about 0.51 equivalent, particularly about 0.5 equivalents relative to Avapritinib.

The solvent in step (b) preferably comprises an alcohol (preferably a C₁ to C₄ alcohol, and more particularly ethanol), acetonitrile, water and mixtures thereof.

In a preferred embodiment, the solvent in step (b) comprises a mixture of ethanol and acetonitrile and/or a mixture of ethanol and water. Preferably, for the preparation of Forms AC1, AC2, AC3, AC4, AC6, AC7, and AC9, the solvent in step may be mixture of ethanol and acetonitrile. Preferably, for the preparation of Forms AC1, AC2, AC3, AC4, AC6, AC7, and AC9, the solvent may be a mixture of ethanol and acetonitrile. Preferably, for the preparation of Forms AC8, AC10 and AC11, the solvent may be a mixture of ethanol and water.

In any embodiment of the process, the ratio of ethanol:acetonitrile or ethanol:water is from: about 3:1 to about 1:3; about 2:1 to about 1:2, about 1.5 to 1 to about 1:1.5 or about 1:1.

In any embodiment, the solvents may be added as a pre-prepared mixture of ethanol and acetonitrile, or ethanol and water.

In any embodiment, the solvent may be used in an amount of: about 0.5 ml to about 50 ml, about 2 ml to about 45 ml, or about 3 ml to about 40 ml per gram of Avapritinib.

The process may comprise milling the mixture of the Avapritinib and solvent in a ball mill. The milling may be carried out at a frequency of about 10 to about 30 Hz, about 18 to about 28 Hz, about 20 to about 28 Hz, or about 25 Hz. The milling may be conducted for about 5 to about 120 minutes, about 10 to about 60 minutes, about 20 to about 40 minutes, or about 30 minutes. In any embodiment, the milling may be conducted at about 15° C. to about 45° C., about 20° C. to about 40° C., about 20° C. to about 35° C., or about 25° C. to about 30° C. The product may be isolated by any suitable method. In any embodiment, the milling vessel is scratched and the product removed from the vessel after scratching.

Alternatively, the process may comprise stirring the mixture of Avapritinib and carboxylic acid in the solvent. The stirring may be carried out at a temperature of about 15° C. to about 45° C., about 20° C. to about 40° C., about 20° C. to about 35° C., or about 25° C. The stirring may be carried out for a sufficient time to prepare the desired product, typically about 8 hours to about 72 hours, about 10 hours to about 48 hours about 16 hours to about 30 hours, or about 24 hours. The product may be isolated by any suitable method, for example, decantation, centrifuge or filtration, preferably filtration.

The product may be dried by any suitable method, for example, in air or in a vacuum oven.

In a further embodiment, the present disclosure includes a co-crystal of Avapritinib HCl salt: benzoic acid; designated Form AHC1.

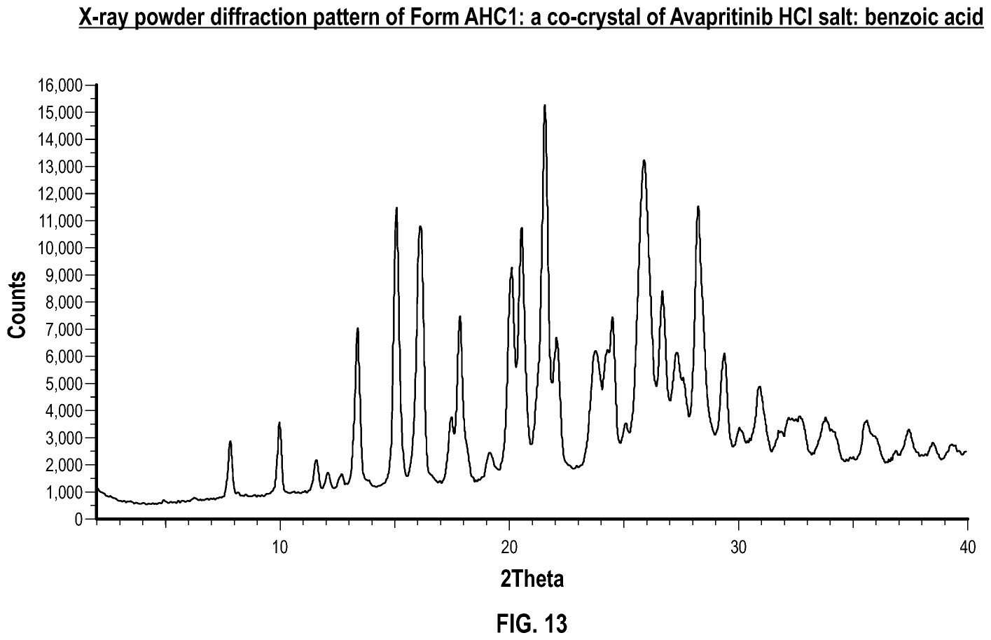

Form AHC1 of the co-crystal of Avapritinib HCl salt: benzoic acid may be characterized by data selected from one or more of the following: an X-ray powder diffraction pattern substantially as depicted in FIG. 13; an X-ray powder diffraction pattern having peaks at 10.0, 13.4, 15.1, 21.6 and 26.7 degrees 2-theta±0.2 degrees 2-theta; and combinations of these data.

Crystalline Form AHC1 may be further characterized by an X-ray powder diffraction pattern having peaks at 10.0, 13.4, 15.1, 21.6 and 26.7 degrees 2-theta±0.2 degrees 2-theta, and also having any one, two, three or four additional peaks selected from 16.1, 20.1, 20.6 and 22.1 degrees 2-theta±0.2 degrees 2-theta.

Crystalline Form AHC1 may be further characterized by an X-ray powder diffraction pattern having peaks at 10.0, 13.4, 15.1, 16.1, 20.1, 20.6, 21.6, 22.1, and 26.7 degrees 2-theta±0.2 degrees 2-theta.

The molar ratio between Avapritinib HCl salt and benzoic acid may be 1:1.

In another embodiment, Form AHC1 may be prepared by stirring Avapritinib: benzoic acid salt with HCl buffer at 25 to 40° C.; preferably 35 to 40° C. Preferably, the HCl buffer is prepared by combining a 0.2 M aqueous HCl solution with a 0.2 M aqueous KCl solution (pH of 1.2). The mixture is preferably stirred for any suitable time to prepare the product. Particularly, the stirring may be carried out for about 8 hours to about 72 hours, about 10 hours to about 48 hours about 16 hours to about 30 hours, or about 24 hours. The product may be isolated by any suitable method, for example, decantation, centrifuge or filtration, preferably filtration. The product may be dried by any suitable method, for example, in air or in a vacuum oven.

The present invention discloses also includes a co-crystal of Avapritinib HCl salt: p-hydroxybenzoic acid; designated Form AHC2.

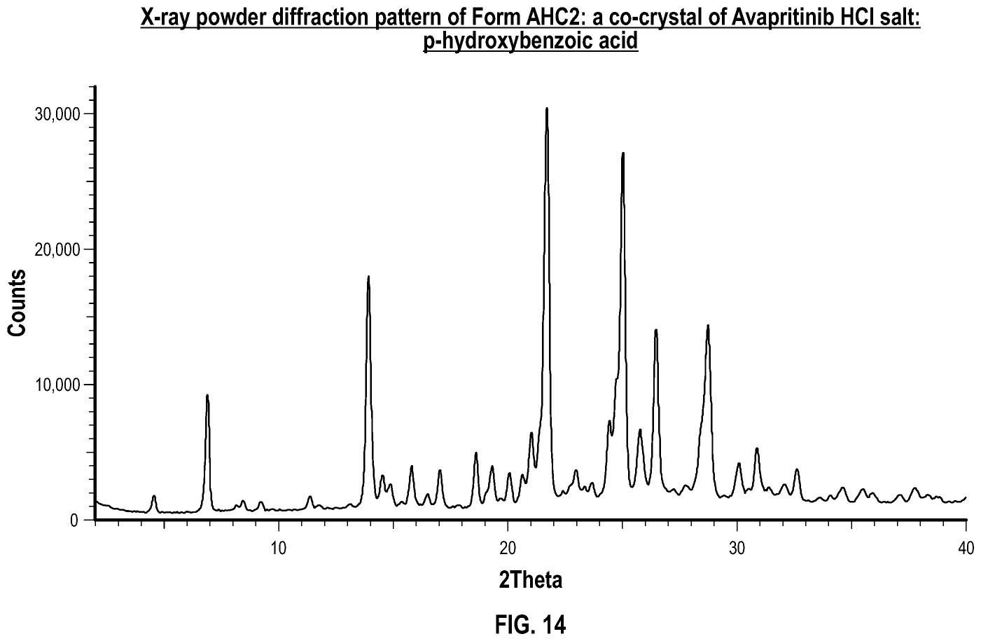

Form AHC2 of the co-crystal of Avapritinib HCl salt: p-hydroxybenzoic acid may be characterized by data selected from one or more of the following: an X-ray powder diffraction pattern substantially as depicted in FIG. 14; an X-ray powder diffraction pattern having peaks at 6.9, 13.9, 21.7, 25.0 and 26.5 degrees 2-theta±0.2 degrees 2-theta; and combinations of these data.

Crystalline Form AHC2 may be further characterized by an X-ray powder diffraction pattern having peaks at 6.9, 13.9, 21.7, 25.0 and 26.5 degrees 2-theta±0.2 degrees 2-theta, and also having any one, two or three additional peaks selected from 19.3, 28.7 and 30.1 degrees 2-theta±0.2 degrees 2-theta.

Crystalline Form AHC2 may be further characterized by an X-ray powder diffraction pattern having peaks at 6.9, 13.9, 19.3, 21.7, 25.0, 26.5, 28.7, and 30.1 degrees 2-theta±0.2 degrees 2-theta.

The molar ratio between Avapritinib HCl salt and p-hydroxybenzoic acid may be 1:1.

In another embodiment, Form AHC2 may be prepared by stirring Avapritinib: p-hydroxybenzoic acid salt; preferably Form AC11, with HCl buffer at 25 to 40° C.; preferably 35 to 40° C. The mixture is preferably stirred for any suitable time to prepare the product. Particularly, the stirring may be carried out for about 8 hours to about 72 hours, about 10 hours to about 48 hours about 16 hours to about 30 hours, or about 24 hours. The product may be isolated by any suitable method, for example, decantation, centrifuge or filtration, preferably filtration. The product may be dried by any suitable method, for example, in air or in a vacuum oven.

The present disclosure also encompasses a co-crystal of Avapritinib HCl: cinnamic acid; designated Form AHC3.

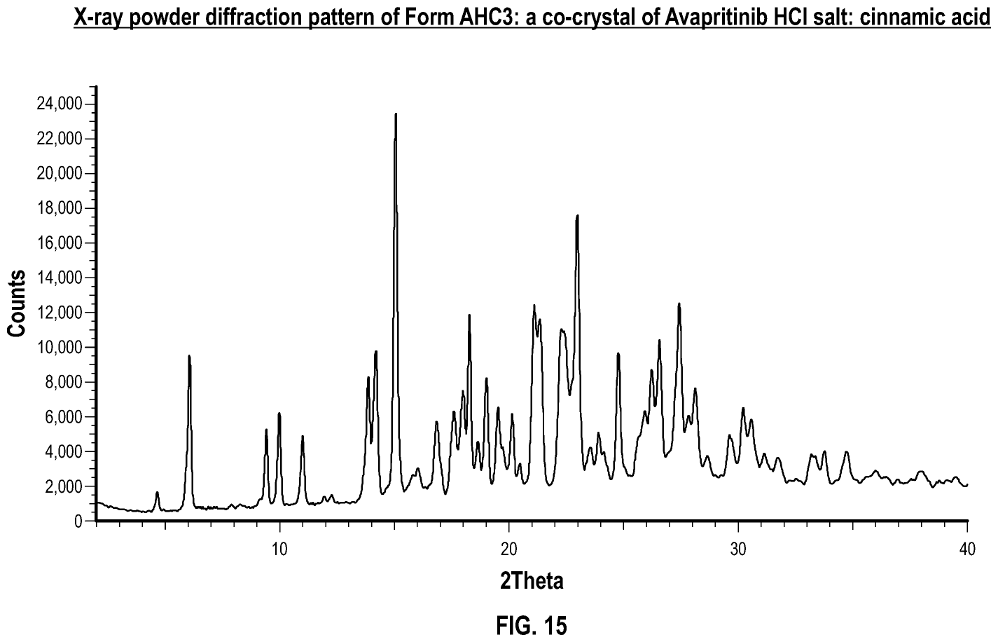

Form AHC3 of the co-crystal of Avapritinib HCl salt: cinnamic acid may be characterized by data selected from one or more of the following: an X-ray powder diffraction pattern substantially as depicted in FIG. 15; an X-ray powder diffraction pattern having peaks at 6.1, 10.0, 23.0, 24.8 and 26.6 degrees 2-theta±0.2 degrees 2-theta; and combinations of these data.

Crystalline Form AHC3 may be further characterized by an X-ray powder diffraction pattern having peaks at 6.1, 10.0, 23.0, 24.8 and 26.6 degrees 2-theta±0.2 degrees 2-theta, and also having any one, two or three additional peaks selected from 13.9, 16.9 and 20.2 degrees 2-theta±0.2 degrees 2-theta.

Crystalline Form AHC3 may be further characterized by an X-ray powder diffraction pattern having peaks at 6.1, 10.0, 13.9, 16.9, 20.2, 23.0, 24.8, and 26.6 degrees 2-theta±0.2 degrees 2-theta.

The molar ratio between Avapritinib HCl salt and cinnamic acid may be 1:1.

In another embodiment, Form AHC3 may be prepared by stirring Avapritinib: cinnamic acid salt; preferably Form AC10, with HCl buffer at 25 to 40° C.; preferably 35 to 40° C. The mixture is preferably stirred for any suitable time to prepare the product. Particularly, the stirring may be carried out for about 8 hours to about 72 hours, about 10 hours to about 48 hours about 16 hours to about 30 hours, or about 24 hours. The product may be isolated by any suitable method, for example, decantation, centrifuge or filtration, preferably filtration. The product may be dried by any suitable method, for example, in air or in a vacuum oven.

In any aspect or embodiment of the present disclosure, any of the solid state forms of Avapritinib as described herein (i.e., Avapritinib:carboxylic acid, or Avapritinib salts), may be polymorphically pure. For example, any of the solid state forms of Avapritinib as described herein may be substantially free of any other solid state (or polymorphic) forms of the subject Avapritinib. Thus, for example, any of the solid state forms of Avapritinib salts as described herein may be substantially free of any other solid state (or polymorphic) forms of the subject Avapritinib salt. For example, the described crystalline Form AC1 of Avapritinib: benzoic acid, particularly Form AC1 of Avapritinib benzoate salt may be substantially free of any other solid state forms of Avapritinib benzoate.

In any aspect or embodiment of the present disclosure, any of the solid state forms as described herein may contain: about 20% (w/w) or less, about 10% (w/w) or less, about 5% (w/w) or less, about 2% (w/w) or less, about 1% (w/w) or less, or about 0% of any other forms of the subject compound, preferably as measured by XRPD. Thus, any of the disclosed crystalline polymorphs of Avapritinib, or Avapritinib salt described herein may be substantially free of any other solid state forms of the Avapritinib, or Avapritinib salt, and may contain greater than about 80% (w/w), greater than about 90% (w/w), greater than about 95% (w/w), greater than about 98% (w/w), greater than about 99% (w/w), or about 100% of the subject crystalline polymorph of Avapritinib or Avapritinib salt. In aspect or embodiment of the present disclosure, any of the described crystalline polymorph of Avapritinib or Avapritinib salt may contain: about 0.5% to about 20% (w/w), about 0.5% to about 15% (w/w), about 0.5% to about 10% (w/w), about 0.5% to about 5% (w/w) about 0.5 to about 2% (w/w), about 1% to about 20% (w/w), about 1% to about 10% (w/w), about 1% to about 5%, about 5% to about 20% (w/w), or from about 5% to about 10% (w/w), of one or more other crystalline polymorph of Avapritinib or Avapritinib salt.

The above crystalline polymorphs can be used to prepare other crystalline polymorphs of Avapritinib, Avapritinib salts and their solid state forms.

The present disclosure encompasses a process for preparing other solid state forms of Avapritinib, Avapritinib salts and solid state forms thereof. The process includes preparing any polymorph according to the present disclosure; or combinations thereof, and converting it to other polymorph of Avapritinib or salt of Avapritinib. The conversion to a salt can be done, for example, by reacting any of the polymorphs of the present disclosure; or combinations thereof, with an appropriate acid, to obtain the corresponding salt.

The present disclosure further encompasses processes for preparing Avapritinib, or solid state forms thereof. The process comprises preparing any of the solid state forms of Avapritinib salts of the present disclosure, and converting it to Avapritinib. The conversion can be done, for example, by a process comprising reacting the obtained Avapritinib salt with an appropriate base to obtain Avapritinib base. The Avapritinib thus obtained may have high chemical purity; preferably above 97%, more preferably above 99%.

The present disclosure provides the above described crystalline polymorph of Avapritinib, including Avapritinib salts for use in the preparation of pharmaceutical compositions comprising Avapritinib, including Avapritinib salts, and/or crystalline polymorphs thereof.

The present disclosure also encompasses the use of the crystalline polymorphs of Avapritinib, including Avapritinib salts, of the present disclosure; or combinations thereof, for the preparation of pharmaceutical compositions of crystalline polymorphs of Avapritinib, crystalline polymorphs of Avapritinib salts, and/or crystalline polymorphs thereof.

The present disclosure includes processes for preparing the above mentioned pharmaceutical compositions. The processes include combining any one or a combination of the crystalline polymorphs of Avapritinib, including Avapritinib salts, of the present disclosure with at least one pharmaceutically acceptable excipient.

Pharmaceutical combinations or formulations of the present disclosure contain any one or a combination of the solid state forms of Avapritinib, including Avapritinib salts, of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Avapritinib and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Avapritinib can be administered. Avapritinib may be formulated for administration to a mammal, in embodiments to a human, by injection. Avapritinib can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Avapritinib, including Avapritinib salts, and the pharmaceutical compositions and/or formulations of Avapritinib, including Avapritinib salts, of the present disclosure can be used as medicaments, in embodiments in the treatment of gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST).

The present disclosure also provides methods of treating gastrointestinal stromal tumors (GIST), solid tumors, and Advanced Systemic Mastocytosis, preferably gastrointestinal stromal tumors (GIST), and solid tumors, and more preferably gastrointestinal stromal tumors (GIST), wherein the method comprises administering a therapeutically effective amount of any one or a combination of the crystalline polymorphs of Avapritinib, including Avapritinib salts, of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

X-ray diffraction was performed on X-Ray powder diffractometer: Bruker D8 Advance; CuK α radiation (λ=1.54 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

Measurement parameters:
Scan range: 2-40 degrees 2-theta;
Scan mode: continuous;
Step size: 0.05 degrees;
Time per step: 0.5 s;
Sample spin: 30 rpm;
Sample holder: PMMA specimen holder ring.
All X-Ray Powder Diffraction peak values are calibrated with regard to standard silicon spiking in the sample.

### EXAMPLES

### Preparation of Starting Materials

Avapritinib can be prepared according to methods known from the literature, for example WO2015/057873.

Starting material Form AT1 can be obtained according to example 1.

Starting material Form AT5 can be obtained according to example 2.

### Example 1: Preparation of Avapritinib Form AT1

Avapritinib (0.2 g) was dissolved in dichloromethane (3 mL) at 25-30° C. in a test tube. The solution was filtered through 0.45 micron filter. The clear solution was covered with paraffin film with a pinhole and kept for slow solvent evaporation at 15-20° C. After 2 days, the obtained solid was analyzed by XRD and designated as Form AT1; as shown in FIG. 11.

### Example 2: Preparation of Avapritinib Form AT5

Avapritinib (AT1, 0.5 g) was dissolved in 1,4-Dioxane (10 mL) under magnetic stirring at 55° C. The obtained hot solution was filtered through 0.45 μm PVDF membrane filter and cooled to 25° C. Diisopropyl ether (50 mL) was separately cooled (0-5° C.) and added to the above clear solution at 25° C. The obtained suspension was cooled to 0-5° C. and stirred for 3 hours. The slurry was filtered at 0-5° C. under vacuum for 15 minutes. The obtained solid was analyzed by XRD and designated as Form AT5 of Avapritinib; as shown in FIG. 12.

### Example 3: Preparation of Avapritinib: Benzoic Acid-Form AC1

Avapritinib (Form AT1) (75 mg) and benzoic acid (18.4 mg) were taken into a 5 mL milling jar, added 2-3 drops of Ethanol: Acetonitrile solvent mixture (1:1 v/v). Added 5 stainless steel balls and ball milled by using Retsch Cryomill instrument at 25 Hz frequency for 30 minutes at 25-30° C. The obtained material was scratched and isolated as free solid which was analyzed by XRPD. The obtained form was designated as Avapritinib: benzoic acid Form AC1; as shown in FIG. 1.

### Example 4: Preparation of Avapritinib: Succinic Acid-Form AC2

Avapritinib (Form AT1) (75 mg) and succinic acid (17.8 mg) were taken into a 5 mL milling jar, added 2-3 drops of Ethanol: Acetonitrile solvent mixture (1:1 v/v). Added 5 stainless steel balls and ball milled by using Retsch Cryomill instrument at 25 Hz frequency for 30 minutes at 25-30° C. The obtained material was scratched and isolated as free solid which was analyzed by XRPD. The obtained form was designated as Avapritinib: succinic acid Form AC2; as shown in FIG. 2.

### Example 5: Preparation of Avapritinib: Fumaric Acid-Form AC3

Avapritinib (Form AT1) (75 mg) and fumaric Acid (8.8 mg)(1:0.5 molar ratio) were taken into a 5 mL milling jar, added 2-3 drops of Ethanol: Acetonitrile solvent mixture (1:1 v/v). Added 5 stainless steel balls and ball milled by using Retsch Cryomill instrument at 25 Hz frequency for 30 minutes at 25-30° C. The obtained material was scratched and isolated as free solid which was analyzed by XRPD. The obtained form was designated as Avapritinib: fumaric acid Form AC3; as shown in FIG. 3.

### Example 6: Preparation of Avapritinib: Adipic Acid-Form AC4

Avapritinib (Form AT1) (75 mg) and adipic acid (11.0 mg) (1:0.5 molar ratio) were taken into a 5 mL milling jar, added 2-3 drops of Ethanol: Acetonitrile solvent mixture (1:1 v/v). Added 5 stainless steel balls and ball milled by using Retsch Cryomill instrument at 25 Hz frequency for 30 minutes at 25-30° C. The obtained material was scratched and isolated as free solid which was analyzed by XRPD. The obtained form was designated as Avapritinib: adipic acid Form AC4; as shown in FIG. 4.

### Example 7: Preparation of Avapritinib: Salicylic Acid-Form AC6

Avapritinib (Form AT1) (75 mg) and salicylic acid (20.8 mg) were taken into a 5 mL milling jar, added 2-3 drops of Ethanol: Acetonitrile solvent mixture (1:1 v/v). Added 5 stainless steel balls and ball milled by using Retsch Cryomill instrument at 25 Hz frequency for 30 minutes at 25-30° C. The obtained material was scratched and isolated as free solid which was analyzed by XRPD. The obtained form was designated as Avapritinib: salicylic acid Form AC6; as shown in FIG. 5.

### Example 8: Preparation of Avapritinib: (R)-Mandelic Acid-Form AC7

Avapritinib (Form AT1) (75 mg) and (R)-mandelic acid (22.9 mg) were taken into a 5 mL milling jar, added 2-3 drops of Ethanol: Acetonitrile solvent mixture (1:1 v/v). Added 5 stainless steel balls and ball milled by using Retsch Cryomill instrument at 25 Hz frequency for 30 minutes at 25-30° C. The obtained material was scratched and isolated as free solid which was analyzed by XRPD. The obtained form was designated as Avapritinib: (R)-mandelic acid Form AC7; as shown in FIG. 6.

### Example 9: Preparation of Avapritinib: Benzoic Acid-Form AC8

Avapritinib (Form AT5, 0.2 gr) and benzoic acid (0.0488 gr) were taken into a test tube. Ethanol (1.25 ml) and water (0.5 ml) were added and the obtained suspension was stirred at 25° C. for 1 day. The slurry was filtered through Whatman filter under vacuum for 15 minutes. The obtained solid was analyzed by XRPD and designated as Avapritinib: benzoic acid Form AC8; as shown in FIG. 7.

### Example 10: Preparation of Avapritinib: Benzoic Acid-Form AC9

Avapritinib (Form AT5, 1.0 gr) and benzoic acid (0.244 gr) were taken into round bottom flask. A mixture of ethanol: acetonitrile (1:1 v/v; 20 ml) was added and the suspension was stirred at 25° C. for 1 hour. Additional portion of ethanol: acetonitrile (1:1 v/v, 20 ml) was added and the suspension was stirred at 25° C. for 1 day.

The obtained slurry mass was filtered through Whatman filter paper under vacuum at 25° C. for 30 minutes. The obtained solid was analyzed by XRPD and designated as Avapritinib: benzoic acid Form AC9; as shown in FIG. 8.

### Example 11: Preparation of Avapritinib: Cinnamic Acid-Form AC10

Avapritinib (Form AT1, 0.1 gr) and cinnamic acid (0.0289 gr) were taken into vial. Ethanol (0.625 ml) and water (0.25 ml) were added and the obtained suspension was stirred at 25° C. for 1 day. The slurry was filtered through Whatman filter under vacuum for 20 minutes. The obtained solid was analyzed by XRPD and designated as Avapritinib cinnamic acid Form AC10; as shown in FIG. 9.

### Example 12: Preparation of Avapritinib: p-Hydroxybenzoic Acid-Form AC11

Avapritinib (Form AT1, 0.1 gr) and p-hydroxybenzoic acid (0.0277 gr) were taken into vial. Ethanol (0.625 ml) and water (0.25 ml) were added and the obtained suspension was stirred at 25° C. for 1 day. The slurry was filtered through Whatman filter under vacuum for 20 minutes. The obtained solid was analyzed by XRPD and designated as Avapritinib: p-hydroxybenzoic acid Form AC11; as shown in FIG. 10.

### Example 13: Preparation of Form AHC1: A Co-Crystal of Avapritinib HCl Salt: Benzoic Acid

Avapritinib benzoic acid (Form AC8, 0.2 g) and HCl buffer (pH 1.2; 5 ml) were stirred at 37° C. for 24 h. The warm suspension was filtered through Whatman filter under vacuum for 15-20 minutes. The obtained solid was analyzed by XRPD and designated as form AHC1: a co-crystal of Avapritinib HCl salt and benzoic acid; as shown in FIG. 13.

### Example 14: Preparation of Form AHC2: A Co-Crystal of Avapritinib HCl Salt: p-Hydroxybenzoic Acid

Avapritinib p-hydroxybenzoic acid (Form AC11, 0.2 g) and HCl buffer (pH 1.2, 15 ml) were stirred at 37° C. for 24 h. The warm suspension was filtered through Whatman filter under vacuum for 15-20 minutes. The obtained solid was analyzed by XRPD and designated as form AHC2: a co-crystal of Avapritinib HCl salt and p-hydroxybenzoic acid; as shown in FIG. 14.

### Example 15: Preparation of Form AHC3: A Co-Crystal of Avapritinib HCl Salt: Cinnamic Acid

Avapritinib cinnamic acid (Form AC10, 0.57 g) and HCl buffer (pH 1.2, 20 ml) were stirred at 37° C. for 20 h. The warm suspension was filtered through Whatman filter under vacuum for 30 minutes. The obtained solid was analyzed by XRPD and designated as form AHC3: a co-crystal of Avapritinib HCl salt and cinnamic acid; as shown in FIG. 15.

1. A crystalline salt of Avapritinib and carboxylic acid, wherein the carboxylic acid is selected from benzoic acid, cinnamic acid or p-hydroxybenzoic acid.

2. The crystalline salt of Avapritinib and benzoic acid according to claim 1, designated as Form AC8, and characterized by data selected from one or more of the following:
a) an X-ray powder diffraction pattern substantially as depicted in FIG. 7;
b) an X-ray powder diffraction pattern having peaks at 8.0, 8.5, 11.2, 14.6 and 31.6 degrees 2-theta±0.2 degrees 2-theta; and combinations of these data;
c) an X-ray powder diffraction pattern having peaks at 8.0, 8.5, 11.2, 14.6 and 31.6 degrees 2-theta±0.2 degrees 2-theta, and also having any one, two or three additional peaks selected from 10.4, 16.5 and 23.5 degrees 2-theta±0.2 degrees 2-theta; and
d) combinations of any a to c.

3. The crystalline salt of Avapritinib and cinnamic acid according to claim 1, designated as Form AC10, and characterized by data selected from one or more of the following:
a) an X-ray powder diffraction pattern substantially as depicted in FIG. 9;
b) an X-ray powder diffraction pattern having peaks at 9.3, 15.6, 22.2, 24.0 and 25.4 degrees 2-theta±0.2 degrees 2-theta;
c) an X-ray powder diffraction pattern having peaks at 9.3, 15.6, 22.2, 24.0 and 25.4 degrees 2-theta±0.2 degrees 2-theta, and also having any one, two, three, four or five additional peaks selected from 4.6, 7.3, 14.9, 17.6 and 19.8 degrees 2-theta±0.2 degrees 2-theta; and
d) combinations of any a to c.

4. The crystalline salt of Avapritinib and p-hydroxybenzoic acid according to claim 1, designated as Form AC11, and characterized by data selected from one or more of the following:
a) an X-ray powder diffraction pattern substantially as depicted in FIG. 10;
b) an X-ray powder diffraction pattern having peaks at 13.0, 13.7, 17.0, 24.6 and 26.3 degrees 2-theta±0.2 degrees 2-theta;
c) an X-ray powder diffraction pattern having peaks at 13.0, 13.7, 17.0, 24.6 and 26.3 degrees 2-theta±0.2 degrees 2-theta, and also having any one, two, three or four additional peaks selected from 8.0, 10.4, 11.7 and 18.7 degrees 2-theta±0.2 degrees 2-theta; and
d) combinations of any a to c.

5. A co-crystal of Avapritinib HCl salt and carboxylic acid.

6. The co-crystal of Avapritinib HCl salt according to claim 5, wherein the carboxylic acid is benzoic acid.

7. The co-crystal of Avapritinib HCl salt according to claim 6, designated as Form AHC1, and characterized by data selected from one or more of the following:
a) an X-ray powder diffraction pattern substantially as depicted in FIG. 13;
b) an X-ray powder diffraction pattern having peaks at 10.0, 13.4, 15.1, 21.6 and 26.7 degrees 2-theta±0.2 degrees 2-theta;
c) an X-ray powder diffraction pattern having peaks at 10.0, 13.4, 15.1, 21.6 and 26.7 degrees 2-theta±0.2 degrees 2-theta, and also having any one, two, three or four additional peaks selected from 16.1, 20.1, 20.6 and 22.1 degrees 2-theta±0.2 degrees 2-theta; and
d) combinations of any a to c.

8. The co-crystal of Avapritinib HCl salt according to claim 5, wherein the carboxylic acid is p-hydroxybenzoic acid.

9. The co-crystal of Avapritinib HCl salt according to claim 8, designated as Form AHC2, and characterized by data selected from one or more of the following:
a) an X-ray powder diffraction pattern substantially as depicted in FIG. 14;
b) an X-ray powder diffraction pattern having peaks at 6.9, 13.9, 21.7, 25.0 and 26.5 degrees 2-theta±0.2 degrees 2-theta; and combinations of these data;
c) an X-ray powder diffraction pattern having peaks at 6.9, 13.9, 21.7, 25.0 and 26.5 degrees 2-theta±0.2 degrees 2-theta, and also having any one, two or three additional peaks selected from 19.3, 28.7 and 30.1 degrees 2-theta±0.2 degrees 2-theta; and
d) combinations of any a to c.

10. The co-crystal of Avapritinib HCl salt according to claim 5, wherein the carboxylic acid is cinnamic acid.

11. The co-crystal of Avapritinib HCl salt according to claim 10, designated as Form AHC3, and characterized by data selected from one or more of the following:
a) an X-ray powder diffraction pattern substantially as depicted in FIG. 15;
b) an X-ray powder diffraction pattern having peaks at 6.1, 10.0, 23.0, 24.8 and 26.6 degrees 2-theta±0.2 degrees 2-theta;
c) an X-ray powder diffraction pattern having peaks at 6.1, 10.0, 23.0, 24.8 and 26.6 degrees 2-theta±0.2 degrees 2-theta, and also having any one, two or three additional peaks selected from 13.9, 16.9 and 20.2 degrees 2-theta±0.2 degrees 2-theta; and
d) combinations of any a to c.

12. The crystalline salt of Avapritinib according to claim 1, which is polymorphically pure, or which is substantially free of any other solid state forms of the Avapritinib product.

13. A pharmaceutical composition comprising the crystalline salt according to claim 1 and at least one pharmaceutically acceptable excipient.

14. (canceled)

15. A process for preparing a pharmaceutical composition comprising combining the crystalline salt according to claim 1 with at least one pharmaceutically acceptable excipient.

16. A medicament comprising the crystalline salt according to claim 1.

17. (canceled)

18. A method of treating gastrointestinal stromal tumors (GIST), solid tumors, or Advanced Systemic Mastocytosis, comprising administering a therapeutically effective amount of the crystalline salt according to claim 1 to a subject in need of the treatment.

19. The co-crystal of Avapritinib HCl salt according to claim 5, which is polymorphically pure, or which is substantially free of any other solid state forms of the Avapritinib product.

20. A pharmaceutical composition comprising the co-crystal of Avapritinib HCl salt according to claim 5 and at least one pharmaceutically acceptable excipient.

21. A process for preparing a pharmaceutical composition comprising combining the co-crystal of Avapritinib HCl salt according to claim 5 with at least one pharmaceutically acceptable excipient.

22. A medicament comprising the co-crystal of Avapritinib HCl salt according to claim 5.

23. A method of treating gastrointestinal stromal tumors (GIST), solid tumors, or Advanced Systemic Mastocytosis, comprising administering a therapeutically effective amount of the co-crystal of Avapritinib HCl salt according to claim 5 to a subject in need of the treatment.
